(19) United States
(12) Patent Application Publication
(10) Pub. No.: US 2020/0204895 A1
(43) Pub. Date: Jun. 25, 2020

(54) Positive Pressure Ventilation Microphone

(71) Applicant: ReddyPort Inc., Salt Lake City, UT (US)

(72) Inventors: Rian Wendling, Salt Lake City, UT (US); Andrew S Hansen, Salt Lake City, UT (US); Joseph Orr, Salt Lake City, UT (US); Richard A Kreifeldt, South Jordan, UT (US)

(21) Appl. No.: 16/805,700

(22) Filed: Feb. 29, 2020

Related U.S. Application Data
(63) Continuation of application No. PCT/US2018/032469, filed on May 11, 2018.
Application No. PCT/US2018/032469 is a continuation-in-part of application No. PCT/US2017/060480, filed on Nov. 7, 2017.
(60) Provisional application No. 62/612,303, filed on Dec. 29, 2017.
(60) Provisional application No. 62/568,314, filed on Oct. 4, 2017.

(51) Int. Cl. H04R 1/08; A61M 16/08; A61M 16/00

(52) U.S. Cl. CPC H04R 1/083; H04R 2410/01; A61M 16/024; A61M 16/0858

(57) ABSTRACT

A microphone module for non-invasive ventilation mask includes a microphone housing that defines and adapter configured to be removably inserted into a port of a non-invasive ventilation mask and form a seal with the port. The microphone module includes microphone elements for generating a speech signal and electrical circuitry for transmitting the mic signal to a cable outside the mask. A microphone system includes an audio processing system and the microphone module connected through a cable. The audio processing system receives the speech signal, amplifies the speech signal and outputs the amplified signal to a loudspeaker.

### CROSS-REFERENCE TO RELATED APPLICATIONS

This application is a continuation of PCT Application No. PCT/US2018/032469, titled Positive Pressure Ventilation Microphone System, filed May 11, 2018, which is a continuation in part of PCT/US2017/060480, filed Nov. 7, 2017, and claims the benefit of U.S. Provisional Application Nos. 62/568,314, filed Oct. 4, 2017, and 62/612,303, filed Dec. 29, 2017. All of the foregoing applications are hereby incorporated herein by reference in their entirety.

### BACKGROUND

### 1. Field of the Invention

The present invention relates to devices and method for providing oral access with a non-invasive positive pressure mask.

### 2. Related Technology

Positive pressure ventilation (PPV) masks are currently used in the medical field for patients with poor oxygen saturation, sleep apnea, and other related respiratory problems. The mask includes a peripheral flexible membrane that contacts the face of the patient and creates a seal with the face using the positive pressure. An example of a positive pressure ventilation mask is disclosed in U.S. Pat. No. 6,513,526 to Kwok. These types of masks used with a ventilator can provide positive pressure airflow, for critically ill patients can do so without the need to intubate the patient or allow earlier extubation.

Positive pressure masks require an effective seal around the facial area and can be a hassle for clinicians or users to properly place. Once in place, the positive pressure in the mask assists the patient's breathing by providing a proper amount of forced air necessary to maintain adequate breathing and exhalation. In a matter of hours or days, the mask can cause discomfort to the patient from dry mouth or nose, nasal congestion, rhinitis or runny nose, facial irritations, bloody noses, dry mucosal tissue, dry lips, increased risk of respiratory infection, or other difficulties managing oral or nasal airway.

Positive pressure masks are also used to treat sleep apnea. While these patients are typically not critically ill, they suffer from the inconvenience of dryness of the airway and the inability to access their oral airway without taking the mask off.

### SUMMARY

The present invention is related to positive pressure ventilation microphone systems. The devices and systems of the invention use a microphone module that includes a microphone element positioned on the inside of a positive pressure ventilation mask to capture the voice of a patient. The microphone can be inserted through an access port of the mask using an adapter or can be integrated into the mask. The voice signal can be processed to detect speech and/or breathing. The detected speech and/or breathing is then used to attenuate noise (e.g., an inhalation noise or exhalation noise), turn off electrical components to conserve battery power (e.g., turn off a power amplifier), and/or set a parameter of a ventilator (e.g., adjust settings for iPAP and/or ePAP).

In some embodiments, the ventilation microphone system processes a speech signal to remove a breathing noise and then the processed signal is amplified and played on a loudspeaker. The loudspeaker can be housed with the power amplifier and batteries that power the loudspeaker. Alternatively, or in addition, the processed speech signal can be outputted to a mobile device such as a mobile phone configured to communicate with a remote person through a voice or messaging service.

Another embodiment of the invention relates to an auto-on feature based on detecting speech. An electrical circuit detects speech activity and turns on components of the microphone system. In some embodiments, the auto-on feature uses an analog speech detector circuit and upon detecting speech in the analog circuit a microprocessor turns on a digital signal processor. The auto-on circuitry can be particularly advantageous for battery powered systems.

Yet another embodiment of the invention relates to a sibilance removal circuit. The sibilance removal circuit analyzes the voice input for excessive loudness of high pitches and upon detecting the imbalance, attenuates high pitches in the audio signal. The sibilance removal can be used with any of the microphone modules described herein, but has been found to be particularly useful for microphone modules configured to be placed in front and near the mouth and/or with the microphone element facing the mouth of the person wearing the mask.

Other embodiments relate to a microphone system using microphone module that has a placement and/or mechanical/acoustical configurations that improve signal to noise ratios, biocompatibility, and/or convenience of using the microphone module in a PPV mask.

For example, in one embodiment, the microphone system can include a module with an adapter and that goes through an access port in the mask and places the mask in front and/or near the mouth while sealing the module with the mask body. The microphone module may include a microphone element with a high sound pressure level and an attenuating material (e.g., dense foam) to reduce the power of sound entering the module.

In yet another embodiment, the module can include a board with microphone elements and the board seals with the housing and the board is coated with an biocompatible material on a side exposed to air from the ventilator.

In yet another embodiment, the microphone module can be removable from an access port in the PPV mask to allow other appliances to be used on the patient through the same access port. The access port may include a valve, preferably a valve that seals under pressure from the ventilator.

### DESCRIPTION OF DRAWINGS

FIG. 1A illustrates a full-face positive pressure ventilation mask, including an elbow connector with a microphone module;

FIG. 1B is a block diagram of a positive pressure ventilator microphone system that includes the mask and microphone module of FIG. 1A;

FIG. 2 is a cross section of the microphone module and mask of FIG. 1A;

FIG. 3A illustrates a microphone enclosure with microphone elements;

FIG. 3B illustrates an alternative embodiment of a microphone enclosure with microphone elements;

FIG. 4 shows another embodiment of a microphone module;

FIG. 5 shows a microphone housing with a removable foam end;

FIG. 6 is a block diagram of a signal processing system;

FIG. 7 is a diagram of digital signal processing for patient activity detection in the DSP of FIG. 6;

FIG. 8A is a circuit diagram of signal analyzer module for processing of FIG. 7;

FIG. 8B is a circuit diagram of the bandpass filter of FIG. 8A;

FIG. 8C is an RC low pass filter of FIG. 8A to adjust timing;

FIG. 8D describes a threshold filter of FIG. 8A;

FIG. 8E illustrates breathing and speech signals in the frequency domain;

FIG. 8F illustrates breathing and speech in the time domain;

FIG. 9 is block diagram of a sibilance removal circuit;

FIG. 10A-10B illustrate an analog filter used in performing an auto-on function;

FIG. 11A is a perspective view of the elbow connector of FIG. 1A;

FIG. 11B is an exploded view of the elbow of FIG. 11A;

FIG. 11C is a cross section of the elbow of FIG. 11A;

FIG. 12A is top perspective view of the self-sealing valve of the elbow of FIG. 11A;

FIG. 12B is a bottom perspective view of the valve of FIG. 12A;

FIGS. 13A-13B illustrates alternative embodiment of a full-face positive pressure mask with an access port; and

FIG. 14 illustrates an alternative embodiment of an elbow with a swivel connector.

### DETAILED DESCRIPTION

The positive pressure ventilation (PPV) microphone systems and modules of the present invention utilize a positive pressure ventilation mask, preferably one with an access port. The access port may be an opening with a removable cap or a valve that can be selectively opened to attach an adapter. The access port may be built into the shell (also referred to as the mask body) of a PPV mask or into a connector (e.g., elbow) of the PPV mask. The valve may be a slit valve or a valve that seals under the pressure of the ventilator (also referred to herein as a self-sealing valve). The valve may also be self-reverting (i.e., made of a material and having a configuration that will revert back to its original configuration when inverted by an object being pulled through the valve). The valve can be positioned in the mask body or in a valve adapter.

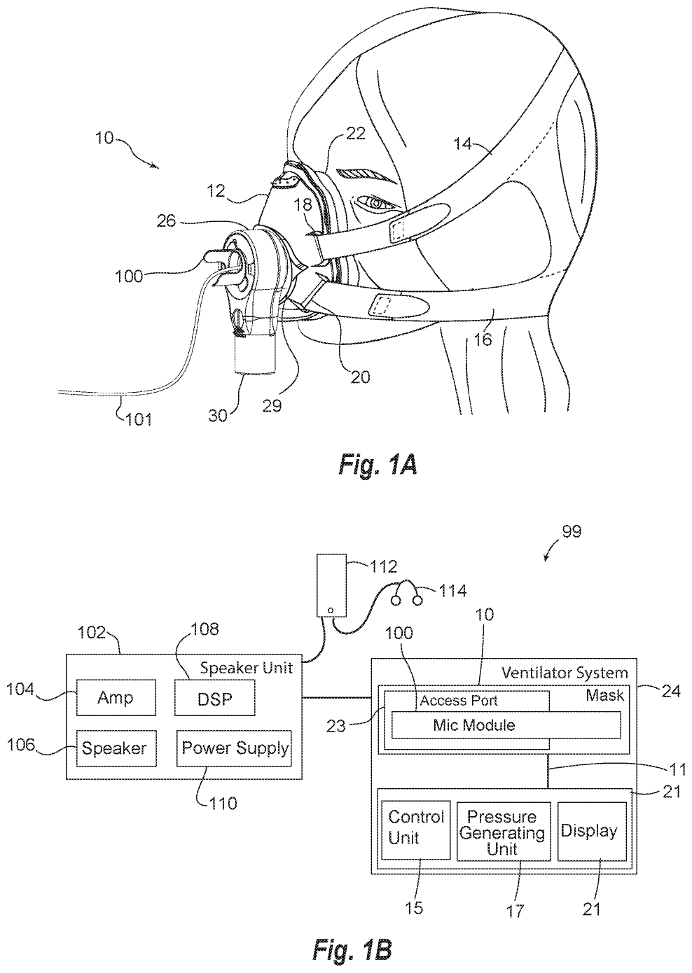

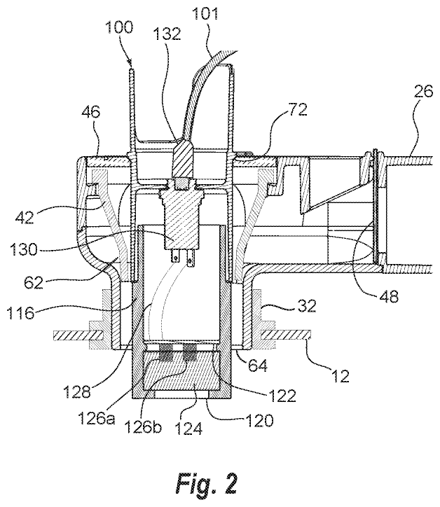

FIGS. 1A and 1B illustrate a positive pressure ventilation (PPV) mask 10 and a voice amplification system 99. The mask 10 includes a mask body 12 (also referred to herein as a “shell”). As shown in FIG. 1B, ventilator system 24 includes the mask 10, access port 23, and a microphone module 100, which includes an adapter that connects to the access port 23 (FIG. 2) and forms a PPV seal with surface 72. Ventilator system 24 also includes a ventilator unit 21 that connects to inlet 30 of the elbow 26 via a flexible hose (not shown) to form a ventilator circuit 11. The ventilator includes a pressure sensor that senses pressure in the system and the sensed pressure is used by control unit 15 to control pressure by driving a pressure generating unit 17 (e.g., an impeller). Parameters of the ventilator can be displayed on display 17 and input received through a user interface (not shown). Ventilators used with the PPV masks of the invention are continuous pressure ventilators and preferably bi-level ventilation is typically important for critical care patients.

Mask 10 is configured to be fluidly coupled to ventilator 21 through air supply connector 26 (e.g., an elbow) and secured to the head of the patient. Mask body 12 can be secured using straps (e.g., upper strap 14 and lower strap 16) or any other suitable securement mechanism suitable for attachment to the head. Straps 14 and 16 connect to eyelets 18 and 20, respectively, on mask body. Straps 14 and 16 connect to eyelets on corresponding locations (not shown) on an opposite side of body 12. The straps secure the mask to the head, which allows a positive pressure seal to be obtained and also avoids movement of the mask relative to the head that could cause air leaks that diminish the positive air treatment.

At the periphery of the mask body 12, mask 10 includes a cushion 22 that includes a flexible membrane (i.e., a flap) that can form a seal with the face of the patient when positive pressure is delivered from pressure generating unit 17 through elbow 26 and into an opening in mask body 12. Cushion 22 forms a seal with the patient's face in a nasal bridge region, a cheek region and a lower lip/chin region of the patient's face. The cushion may be constructed of one or more relatively soft, elastomeric materials connected to the mask body, which is typically constructed of a second material (or the same material but thicker) that is more rigid than the cushion. The cavity of mask body 12 forms a positive air pressure chamber between it and the face of a person. For purposes of this invention the term “within the mask” means the chamber defined by the mask when on the face of a person.

Masks having membranes suitable for sealing around the mouth and nose of a patient using positive pressure are described in U.S. Pat. No. 9,119,931 to D'Souza, U.S. Pat. No. 9,295,799 to McAuley; U.S. Pat. No. 6,513,526 to Kwok, and D464,728 to Paul, and international application publication WO2017021836A1 to Rose, all of which are hereby incorporated herein by reference. The mask may also include an exchangeable two mask system such as the FDA cleared AF541 mask by Respironics (Murrysville Pa., USA) and masks with similar features and function.

Microphone module 100 includes one or more microphone elements, preferably at least two microphone elements, and is electrically coupled to an audio processing system, which may be part of a speaker unit 102 (or alternatively ventilator system 24). Speaker unit 102 includes a digital signal processor 108, a power supply 110, and a power amplifier 104 that outputs to a loudspeaker 107. Alternatively, the audio from the audio processing system can output to a communications device 112 and/or headphones 114. The communication between mic module 100 and speaker unit 102 and/or communications device 112 may be a hard wire or wireless. The mic module 100, speaker unit 102 or communications device 112 can include a transceiver configured to establish a wireless connection and transmit and/or receive audio data.

Loudspeaker 102 may be placed bedside. A bedside speaker can be advantageous because the sound appears to come from the patient, which will sound more natural. More natural speaking can be important to critically ill patients since they are frequently at end of life and desire to communicate with loved ones for the last time. In addition, providing a patient with voice amplification can be important for compliance and patient comfort. If the patient can hear themselves talk, they can relax because the sound is as expected. If the sound is muffled the patient naturally tries harder to make more sound even if such effort is not necessary.

FIG. 2 is a cross section of a portion of mask 10 and mic module 100. FIG. 2 shows an adapter 116 that has an extension housing 118 that extends the housing of the adapter in the distal direction (i.e., toward the oral end). At the oral end, the extension housing 118 has an opening 120 that faces the mouth of a person when the adapter is positioned in mask 10.

Adapter 116 and its extension housing 118 can disconnect for placing components of the microphone module into the housing and assembling the housing around the microphone module components (e.g., a press fit or a snap connect). The housing is preferably configured to place the microphone within the cavity of the mask close the patient's mouth, which has been found to be important in some embodiments for obtaining suitable signal to noise ratio for performing accurate digital signal processing.

The length of extension housing 118 is selected to place the opening near the mouth. Preferably less than 3, 2, 1.5, or 1 inch and/or greater than 0.25, 0.5 or 1.0 inch and/or within a range of the foregoing. The length of the adapter and housing as measured from the opening of the access port to the oral end of the microphone module may be greater than 1, 1.5, 2, 2.5, 3 inch and/or less than 6, 5, 4, 3.5, 3, 2.5, or with a range of the foregoing.

Adapter 116 is positioned in the access port which extends from ring 46 and opening 64 of elbow 26. Microphone module 100 extends through opening 64 so as to place the microphone beyond mask body 12 and its adjacent structure, swivel 32. Moving the microphone out of the opening 64 and/or away mask body 12 (i.e., the shell) has been found to substantially improve the signal to noise ratio. In one embodiment, the adapter is configured to couple with the access port and place the opening 120 to the microphone at least 0.25, 0.5, 0.8, or 1.2 inch inside the mask from the center point of opening 664 (i.e., the inside opening of the access port). The opening 120 of microphone module is preferably facing a mouth region of the person so as to receive direct sound from speech from the mouth.

A plurality of microphone elements 126a and 126b (collectively 126) are mounted on circuit board 122. Microphone elements 126 can be an electret or a MEMS. The microphone element may be a condenser mic and/or require phantom power. The phantom power may be in a range from 2-10 volts, preferably 3-5 volts. Electrets can be preferred for their high sound pressure levels, which has been found to be important in the NIV Mask environment. MEMS can be preferred for minimizing size of the module and availability of bottom firing elements. The microphone element may be an omnidirectional microphone or a directional microphone. Preferred elements have a high dynamic range and/or high sound pressure level. Digital MEMS (a/d converter on mic board) are also suitable, which can be used to reduce electrical noise from hospital equipment placed near the bedside. Digital MEMS may also be useful for having more microphone elements with fewer wires since the signals from different elements can be transmitted on the same wire. In some embodiments, the microphone element may be an active mic (power sent to the mic). The microphone element may also have its own pre-amp before the preamp in the audio processing system. A pre-amp on the microphone can reduce clipping of the microphone, which can be a particularly difficult problem with voice amplification on positive pressure masks due to the increase in pressure. Although not preferred, some embodiments can use a single microphone element. Noise cancellation with a single microphone element can require additional computation power. Noise cancellation can be performed using the frequency domain to identify non-speech elements of the signal.

Preferred embodiments of the system use two or more microphone elements. The two or more elements can perform processes upon coincident signals are useful, such as in discriminant noise cancellation. Two microphone elements may be mounted on a board and/or within housing. The microphone elements may be differently specified microphone elements or preferably identical specification mic elements. The mic elements may be mounted in the same plane, off plane, and/or at different angles. Same plane microphones may facilitate manufacturing while differently angled microphones may provide better discernment of off-axis signals. Detecting off-axis signals can facilitate detecting incoherent (e.g., turbulent) sounds as opposed to coherent.

In a preferred embodiment, the microphone has a relatively high max sound pressure level. The closeness of the microphone in the mask and the relatively high pressure in the mask causes surprisingly high sound levels even for patients talking moderately loud or quietly. The microphone module may include a sound attenuating material create an effective sound pressure level that avoids microphone clipping for a person talking at 50, 60, or 70 dB. For purpose of this invention, unless otherwise indicated, effective sound pressure level is the sound pressure level of the microphone plus the decibels by which the sound attenuating material attenuates. The sound attenuating material may have a thickness and/or a density that prevents clipping of a microphone in the housing when placed in the mask. The sound attenuation of a foam may depend on its density and thickness.

An example of a suitable electret may have the following specifications plus or minus 5%, 10%, or 20% for any: −42±3 dB RL=2.2 kΩ Vcc=2.0 v (1 kHz 0 dB=1 v/Pa) Impedance Max. 2.2 kΩ 1 kHz (RL=2.2 kΩ) Frequency 50-12000 Hz Current Consumption Max 0.5 mA Operating Voltage Range 1.0-10 V Max SPL (dB) 120 dB S/N Ratio More than 58 dB Sensitivity Reduction 2.0−1.5V Variation less than 3 dB Storage Condition −20˜+60° C.; R.H.<45%-75% Operating Condition −10˜+45° C.; R.H.<85%.

In a preferred embodiment the microphone element has a diameter less than 0.8, 0.5, 0.3, 0.25, 0.2, 0.15 and/or greater than 0.03, 0.05, 0.1, or 0.15 inch and/or within a range of the foregoing. The microphone elements may be a directional microphone or an omni directional. Microphone elements 126 are selected to have a low self-noise, a high max sound pressure level (“SPL”) (also known as acoustical overload point), and/or a high dynamic range and/or a small size. For purposes of this invention, the SNR is measured with a standard reference pressure of 94 dB SPL (1 Pa) at 1 kHz. In one embodiment, the dynamic range is at least 80 dB, 85 dB, 90 dB, or 95 dB, the SNR is at least 60, 65, or 70 dB and/or the max SPL of the microphone element is at least 80, 85, 90, 95, 100, 105, 110, 115, 120 and/or less than 160, 150, 140, 130 or within a range of any of the foregoing endpoints (at the conditions set forth above for the suitable electret).

Module 100 can include an attenuator 124. Placing the microphone close to the patient's mouth can cause excessive gain or clipping of the microphone. To reduce the power of the vocalization, a sound attenuating material can be placed between the mic elements and the mouth of the patient. The sound attenuating material may be a dense or thick foam. A high dynamic range microphone placed near the mouth and attenuated can produce a signal that is suitable for processing in a digital signal processor. In one embodiment the attenuator may be a foam with a density of at least 2, 2.5, 3, 4, or 5 lb ft3 or less than 10, 8, 7.5, 7, or 6 lb ft3. In a preferred embodiment, attenuator is a biocompatible foam. Traditional foam windscreens typically have a density less than 2 lbs ft3, has been found to not be sufficient to attenuate the power of the voice when using a high dynamic range or high max SPL mic placed near the mouth in a PPV mask. In one embodiment, the attenuator reduces the sound pressure level across the attenuator by at least or less than 3, 5, 10, 12, 15, or 20 dB or a range thereof.

Wires 128 connect board 122 with jack 130. Jack 130 is mounted to the body of adapter 116 and is in electrical communication with cable connector 132 and cable 101 is inserted into jack 130 and extends in the proximal direction away from jack 130. Jack 130 may form a PPV seal with adapter to maintain pressure in mask 10. Alternatively, cable 101 can be mounted in adapter 101 and electrical coupled to mic elements 126 inside adapter 116. Or as described below, the seal can be between board 122 and extension 118 of adapter 116.

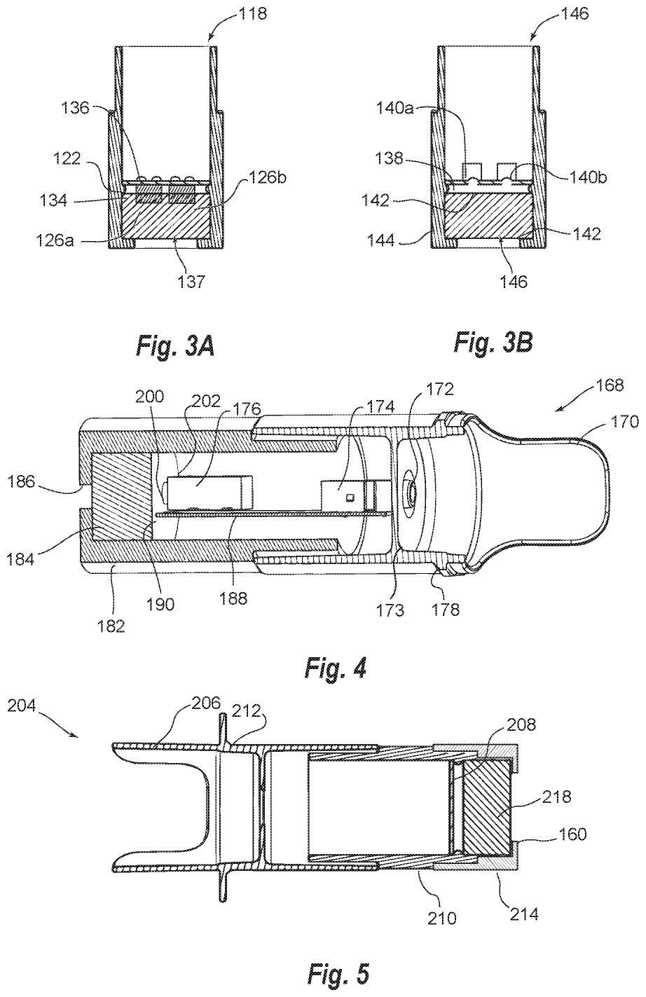

In some embodiments, most or all of the electrical components are isolated from the distal opening of the microphone housing to prevent ventilation gases from reaching the isolated electrical components. FIG. 3A shows an extension housing 118 with a cavity 137 bounded by circuit board 122 and walls of extension 118. Cavity 137 has an opening 120 at the oral end. Microphone elements 126a and 126b are disposed within cavity 137 as well as an attenuator material. Circuit board 122 can be sealed to annular feature 134 on the wall of extension housing 118. Any technique can be used to form the seal including press fit, heat welding adhesion, snap connects and any other connection suitable for use with connecting a board to a housing. Cavity 137 may be coated with a biocompatible polymer prior to or after mounting microphone elements 126a and 126b. Microphone elements can be connected with pins that are soldered to form solder bumps 136.

FIG. 3B illustrates an embodiment of a sealed microphone cavity similar to FIG. 3A but with bottom firing microphones. Board 138 is mounted or sealed to housing of extension 148 to form cavity 146. Microphone elements 140a and 140b are mounted on the proximal side of board 138 opposite cavity 148 and opening 120. Holes (e.g., hole 142) are formed in board 138 to allow sound entering opening 120 to pass through board 138 and into the bottom of elements 140. Microphone elements 140 may be flow soldered to board 138 prior to being secured in extension 146. Cavity 146 may be sealed with a biocompatible coating prior or after mounting bottom mount microphone elements 140a and 140b.

FIG. 4 illustrates yet another alternative embodiment of a microphone module 168. Module 168 includes an adapter 170 with an extension housing 182 attached thereto. Adapter 170 includes a seal structure 178 configured to connect to and seal with a port 23 in mask 10. Module 168 includes an opening 186 at an oral end thereof and an attenuator 184 disposed within housing 182. A microphone element 200 is mounted facing opening 186 using a connector 176. Board 188 extends from a distal end to wall 173 of adapter 170. Board 188 can be used to avoid using wiring between mic elements and the connector 174 in wall 173. Connector 174 can include a jack 172 for attaching a cable. Module 168 can have a wall (not shown) that originates at point 202 and extends transverse and around element 200 to provide an aperture to seal element 200.

FIG. 5 describes yet another embodiment of a module 204 that includes an adapter 206 with a seal structure 212 and a housing extension 210 and a board 208. A removable cap provides access to attenuator 218 to make it easily replaceable. Replacing attenuator 218 or a foam can be advantageous to avoid harboring bacteria. This can be advantageous in a critical care setting where infections are particularly challenging for patients to recover from. The cap can have a press fit, threads or any other mechanism suitable for connecting the cap to the housing.

In some embodiments, the audio processing system, speaker, and battery power can be built into the housing of the microphone adapter to avoid having cords or other elements attached to the patient. This embodiment is preferred where small speakers and limited power are suitable and where cords are particularly problematic. In other embodiments, the microphone module connects to a speaker housing and/or amplifier housing including the amplification and signal processing components.

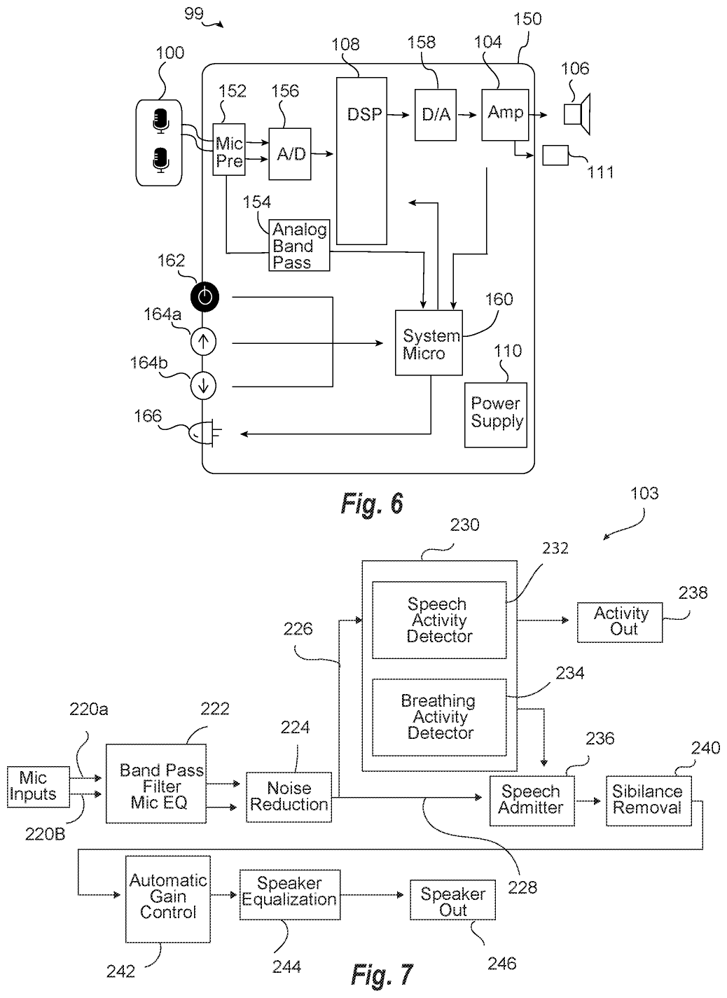

FIG. 6 illustrates an example hardware design for an audio processing system 150 suitable for use in voice amplification system 99. System 150 includes a microphone preamplifier 152, an analog band pass filter 154, an analog to digital converter 156, digital signal processor 108, digital to analog converter 158, power amplifier 104, system microprocessor 160, power supply 110, power on/off switch 162, volume up and down buttons 164a and 164b, power indicator LED 166, and an signal output 111.

System 150 receives at least one microphone input from microphone module 100 (preferably a plurality of microphones inputs). Each audio signal is gain adjusted in microphone preamp 152 and then converted to a digital signal using analog to digital converter 156 and/or output to analog band pass 154. The digital signal is then processes using digital signal processing 108 and converted back to analog using digital to analog converter 158. The processes analog signal is amplified using power amplifier 104 and output to a loudspeaker 107 or another communication device 112, such as desktop computer, laptop computer, mobile phone, or the like.

The communication device 112 may include communication software that initiates a phone call or messaging service to transmit the processed voice signal to a remotely located person, thereby allowing communication between the patient and the remotely located person. The communication software may include a voice to text converter for converting a patient's voice to text and communicating the text to the remotely located person.

Analog band pass can be used for detecting voice signals and the detected voice signals can be used by system microcontroller 160 to turn on components of the system (e.g., DSP 108).

All or portions of the components of system 150 may be housed in a stand-alone enclosure, the microphone module 100, a speaker enclosure (e.g., speaker unit 102 (FIG. 1B), communication device 112, headphones 114, ventilator unit 21 (FIG. 1B), or combinations of these (e.g., pre-amp in module 100, DSP 108 in communication device 112 or in ventilator unit 21).

System microcontroller 160 can be used to receive input from volume buttons 164 and/or power button 162. The system microcontroller can provide output such as through LED 166 to indicate the state of the system or other information (e.g., on, off, standby, voice detected, an error, low battery, adequate battery, etc.)

FIG. 7 is a flow diagram 103 illustrating audio signal processing 103, which can be carried out for example on DSP 108 (FIG. 1B). The audio signal from mic inputs 220a and 220b are each processed using a band pass filter and frequency equalizer 222. The band pass filter can be used to cut out frequencies outside speech. In some embodiments, the band pass can filter out frequencies less than 300, 250, 200, 150, or 100 and/or greater than 3000, 3400, 4000, or 5000, and/or within a range of any of the foregoing. The mic EQ can be used to correct non-flat microphone response. In one embodiment, the mic EQ is used to correct a non-flat signal caused by an NIV mask. The frequencies passed by the band pass filter may be equalized to produce a more even sound across the range of frequencies. The frequency equalization typically corrects variation created by the microphone elements and is often specific to the particular microphone being used and its configuration in the mask housing mask.

The audio signal is then processed using noise reduction block 224. The noise may be incoherent (e.g., turbulent) sounds produced within the PPV mask or sounds that are off axis from the speech sound. Incoherent sounds typically include wind noise (created in the mask), ventilator noise (piped down tube or vibration from outside mask), ambient noise (beeps, alarms, non-voice ambient sounds) self-noise (noise floor or white noise). Noise reduction block can compare mic inputs 220a and 220b and detect the amplitude and phase. Sound that is out of phase and has a high amplitude can be indicative of noise generated from incoherent sounds. The incoherent sounds are then attenuated to remove the noise from the audio stream. Out of phase noise is best detected using dual microphones (preferably identical microphones) positioned spaced apart and near each other and positioned in the same plane. Alternatively, and less preferred, a single microphone signal can be analyzed for characteristics that are indicative of incoherent or turbulent noise (e.g., analyzing frequencies and/or envelope characteristics), which is then used to attenuate the noise.

The audio stream generated in an NIV mask has been found to have noises that cannot be eliminated using the foregoing dual mic noise reduction techniques. NIV masks can create breathing noises (inhalation and exhalation) where a significant portion of the noise is coherent with speech and/or are non-turbulent. These breathing noises have a pronounced un-natural sound (similar to the breathing by Darth Vader in the movie Star Wars). The present invention relates to audio processing systems that can remove these noises using a patient activity detector. Digital signal processing 103 includes an activity detector that detects patient activity such as breathing or speech and then uses the detected patient activity to attenuate the noise. The DSP patient activity detector can also be used to reduce power usage in the microphone system and/or to adjust settings on the ventilator.

A side stream 226 of the audio stream is processed in patient activity detector 230. Activity detector 230 can include speech activity detector and/or breathing activity detector 234. Side stream 226 splits from main audio signal 228 so that activity detector 230 can remove portions of the speech to detect the activity even if removing those portions of the stream are also important for maintaining a natural sounding voice (i.e., the main audio stream retains the portions of the speech removed for activity detection). The output from activity detector 230 is then used by speech admitter 236 to selectively pass the main audio signal 228. Alternatively, the detected activity may be used by system micro 160 (e.g., to power down amplifier 104) or outputted through activity out 238 to a ventilator 21 or other device.

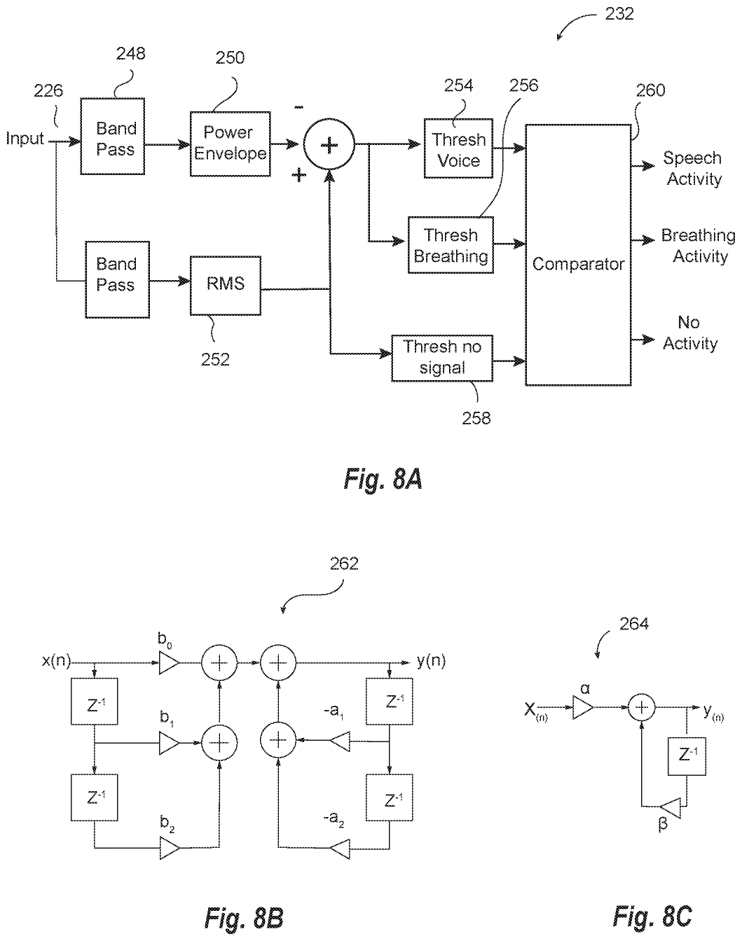

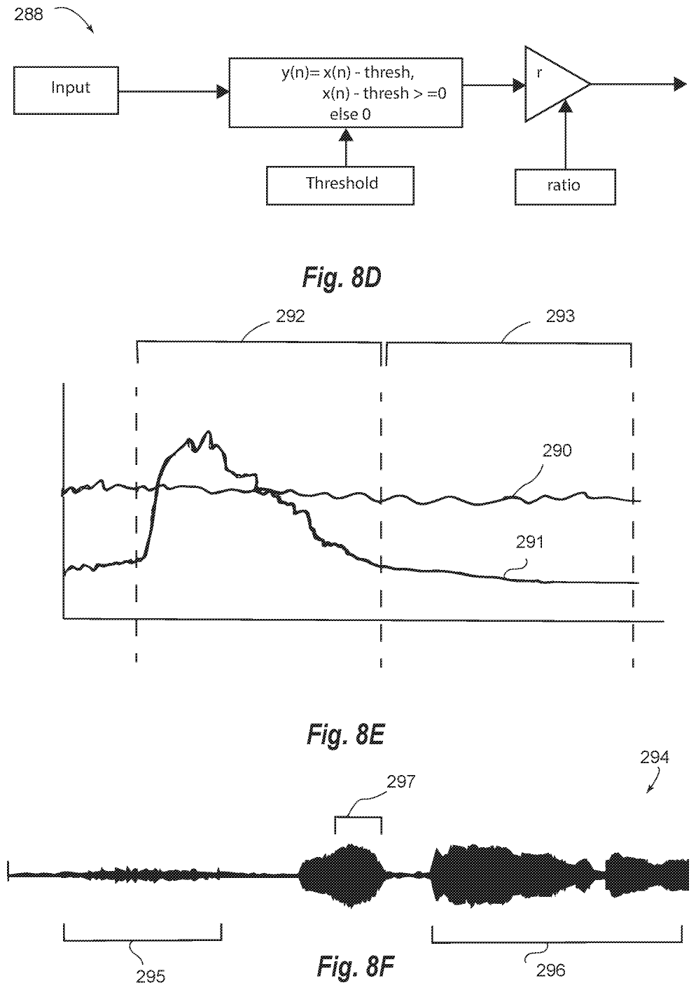

FIG. 8A illustrates an example circuit for performing activity detection according to one embodiment of the invention. Side stream 226 is split and processed using band pass filter 248 and band pass filter 249. Band pass filter 248 aggressively filters out portions of the voice data and passes particular frequencies that are indicative of speech and breathing (breathing in and/or breathing out). The band pass filters can be the same or different. The band pass filters can be an infinite impulse response filter or a finite impulse response filter. The IIR filter may be a direct form I or II, preferably a direct form I as shown in FIG. 8B (circuit 262). Band pass is performed using a cascade of at least two of the direct form I filters shown in FIG. 8B. In a preferred embodiment, b0, b1, b2, a1 and a2 are 2nd order Butterworth filter. In some embodiments, the band pass filter is an at least second order filter, more preferably at least 4th order filter or a 6th order filter. The band pass filter is used to produce a frequency of interest. For example, band pass filter can be used to attenuate frequencies that are outside a frequency range of interest for detecting speech or breathing. The band pass filter may be placed before power envelope 250, RMS 252, or both, or other components of the circuit. The band pass filter can include a plurality of band pass filters and/or split the audio stream and perform on two or more portions of the audio stream. The activity detector may filter out parts of the speech signal to isolate frequencies that are unique to speech and/or unique to breathing. In some embodiments, the band pass filter of the activity detector is configured to attenuate all or a portion of the speech frequencies less than 150, 250, 300, 400, 500, 1000, or 2000 hz and/or greater than 5000, 4000, 3000, 2000, or 1000 hz, or within a range of the foregoing endpoints. In some embodiments, the bandpass filter removes the first harmonic, or second, more preferably the third or fourth harmonics and passes the speech fundamental and/or the first harmonic. In some embodiments the bandpass removes the fundamental frequency and lower harmonics and passes only the upper harmonics.

Once side stream 226 has been processed in the band pass filter, signal 226 is then processed in power envelope block 250 and root mean square block 252. The RMS is a long-term average power of the signal. The power envelope is a short-term average power of the signal. The power envelope is somewhat equivalent to a smoothing of the envelope. FIG. 8C describes a digital RC filter 264 that can be used in the present invention to determine power envelope 250 and/or RMS 252. The RC filter 264 may be an integrator circuit where alpha+beta=1 and if X(n)>y(n), alpha is =alphal B=B1 else alpha=A2, beta=B2. For the power envelope 250, the alpha coefficient is set high (e.g., greater than 0.5, 0.8, or 0.9) and for the RMS 252, the alpha coefficient is set low (e.g., less than 0.5, 0.2., or 0.1). RMS 252 can use a plurality of RC circuits having different coefficients to achieve a stronger signal for a given average time.

FIG. 8D describes a threshold filter 288 that can be used in the present invention to select a threshold. For example, threshold filter 288 can be used for the threshold of voice 254, threshold of breathing 256, or threshold of no signal 258 as shown in 8A.

The difference between power envelope 250 and RMS 252 in combination with the RMS produces three states that indicate breathing noise, speech, or no activity, respectively. If the difference between power envelope and RMS is high, and the RMS is high, then threshold for voice will be greater than zero and the threshold for “no signal” is 0, which is indicative of speech. If the difference between the power envelope and RMS is low and the RMS is high, then the threshold for breathing is greater than zero and the threshold for “no signal” is zero, which is indicative of a breathing noise. If the RMS is low, then threshold for no signal is greater than zero, which is indicative of no signal. The signals for speech, breathing, and no activity can be passed through a threshold circuit to allow probabilities to be associated with each signal prior to being integrated in comparator 260. The comparator receives the signals from the threshold circuit and produces the returned state of the patient activity (speech activity, breathing activity, or no activity).

The alpha and beta coefficients used for the power envelope 250 and RMS 252 can be selected to set a time average of the signal. In one embodiment, the power envelope is averaged over a period of time of at least 0.25, 0.5, 1, 5, 10 ms and/or less than 30, 20, 10 ms, or with a range of any of the foregoing endpoints. In some embodiments, the RMS of the signal is averaged over a period of time greater than 3, 5, or 10 ms and/or less than 250, 100, or 50 ms, or with a range of any of the foregoing endpoints. In some embodiments, the time average for the RMS is at least 3, 5, 10 times greater than the sampling time for the power envelope and/or less than 250, 100, or 50 times the sampling time for the power envelope, and/or within a range between any of these endpoints.

Admitter 236 has a gain element that opens or closes to attenuate or pass signal 228 through admitter 236. When speech is detected the gain element opens and signal 228 passes. When no speech is detected or when breathing noise is detected, the gain element closes and signal is blocked. Admitter 236 may have a small delay to allow for speech detector to process the signal. Delay is preferably less than 40, 20, 10, or 1 ms. Admitter may have a ramp between beginning and ending changes in the level of attenuation. The ramp may be less than 20, 15, 10, 5, or 1 ms and/or greater than 0.01, 0.05, 1 ms, and/or within a range of the foregoing endpoints. The ramp may be exponential or linear. Ramping can be important for naturally sounding speech. If ramping is too slow, the sound will be chopped off. If ramping happens too quickly, the sound can pop.

In an alternative embodiment, speech admitter 236 can receive a breathing activity signal from ventilator 21. In this embodiment, a pressure sensor in the ventilator circuit detects negative pressure indicative of inhalation. The negative pressure activity can be transmitted to audio processing system 150 and used by admitter 236 to attenuate the main audio signal 228. Speech admitter 236 may use the breathing activity from a pressure sensor alone or in combination with other breathing activity.

FIG. 8E illustrates a breathing signal 290 and speech signal 291 in the frequency domain (x axis is frequency and y axis is power). As shown, the breathing noise tends to be wide band and has similar power in a low band 292 and a high band 293. Breathing can be detected using low pass filtering and comparing it to a wide band signal. Since the harmonics of speech have less power, more power will be detected in the low pass filter as compared to the high pass filter for speech. Speech vs. breathing can be detected because if the signal is speech, the wide band will be not much more or similar to the low band pass. If breathing, the wide band will have substantially greater power than the low band pass.

In another embodiment, a high band pass filter is used with a wide band signal. In this embodiment, breathing is detected if the high band pass signal is similar to the wide band filter. Voice is detected if the high band pass filter is substantially less than the wide band filter.

In yet another embodiment, the low pass filter signal can be compared with the high band pass filter. If the signal is breathing, the high band pass filter will be substantially less than the low band pass filter. If the signal is speech, then the power for the high band pass signal should be substantially less than that of the low band pass filter.

The speech activity detector or breathing activity detector may use methods other than a comparison of power envelope and RMS to detect speech and/or breathing activity. In order for the detection to be fast it can be advantageous to perform signal processing that is based on crest factor features. Speech tends to have a high crest factor and breathing noises tend to have a low crest factor. FIG. 8F shows a time domain signal 294 with a breathing noise 295 and speech 296. The breathing noise produces a longer flatter signal 295 as compared to speech 296. The flatter signal can be identified by its crest factor, which will be lower over the short term and long term as compared to speech. Speech on the other hand may have a high crest factor. The envelope of speech changes rapidly over short periods of time whereas breathing noise has an envelope that changes more gradually over the same amount of time. In some embodiments, processing includes calculating the number of times in a given period that the signal is above a threshold and setting a parameter for when the number of times within a period or the number of times in a row that the threshold is reached is indicative of the patient activity. In a preferred embodiment, the process is carried out in the digital domain. In some embodiments, the threshold is advantageously determined in the log domain to reduce dynamic range and discriminate the signal in the threshold filter. In some embodiments, the crest factor feature can be used to distinguish between breathing and non-vocalized consonants such as the “wh” sound or “sh” sound. These sounds can appear similar to a breathing noise over a short period of time in the time domain. To prevent false positives for breathing, the signal may be analyzed for crest factor. If the signal has a low crest factor it is likely breathing signal and if the crest factor is high, speech. The crest factor feature may be used alone or in combination the features shown in FIG. 8A. Preferably band pass filtering is performed first and if breathing is detected a second DSP processing step is performed using the crest factor feature to remove false positives (i.e., if first filtering detects breathing, a second DSP processing step is performed.

In yet another embodiment, breathing noise and/or speech can be detected using a threshold filter with adjustable gain (e.g., buttons to allow the threshold to be adjusted up and down). The threshold filter may compare an upper cutoff to a lower cutoff and if the high pass portion of the filter is greater than the low pass portion the signal is likely breathing. Adjusting the threshold up can be useful for female voices, which tend to be higher and adjusting the threshold lower allows the system to be optimized for a male or lower voice.

In some embodiments, the digital signal processing to detect speech and/or breathing noise can include creating a plurality of filter banks over the range of speech frequencies. For example, a series of band pass filters paired with an RC circuit (FIGS. 8B and 8C) can be used to create filter banks at different frequencies. Increasing the number of filter banks allows increased discrimination between sounds that may be voice or breathing noise. Filter banks at frequencies in the 1200-3500 Hz range can be particularly advantageous to distinguish between speech and noise because speech in that range should be diminishing. Increases in signal strength in that region is indicative of noise. Typically, the number of filter banks can be between 2-20 or more. For larger numbers of filter banks (i.e., to achieve narrower bands of frequencies) Fast Fourier Transform of a particular number of points can substitute for filter banks.

Importantly, patient activity detector is not based on speech recognition. Speech recognition requires determining what was said, not just whether a human voice is active. Speech recognition would cause a delay that would require chopping the speech signal in the admitter or cause an unacceptable delay that would be perceptible to the user (e.g., greater than 80 ms). The sound being amplified in the present invention is used for communication and therefore is desirably a natural sounding voice.

Filtering breathing noise has been found to be particularly important for communication with a ventilated patient. PPV masks create problematic breathing noises that are not a problem in other settings such as pro audio. The breathing noises in the mask can be distinguished from speech based on their frequency and envelope patterns (e.g. crest factor). The breathing noise in PPV masks is substantially different compared to noises found in typical noise cancelling devices such as Bose headphones. In those devices, the noise is wide band (e.g., similar to a jet engine or shhhhhhh) and has narrow tones (e.g., constant single frequency). Breathing in an PPV mask is neither. Breathing has a shifting frequency content (shifting high low or low high) and complex tones.

Controlling Ventilator Based on Detected Activity. Some embodiments of the invention relate to controlling the ventilator using detected activity. For example, where speech is detected, the duration of the speech can be used to time iPAP in a bi-level ventilator. In some embodiments, the audio signal is collected while the patient is at therapeutic levels of ventilator pressure and the audio stream is processed on audio that was collected at therapeutic levels (ePAP, iPAP, or both).

Some embodiments of the invention relate to producing a natural sounding voice from a PPV patient by adding missing harmonic content. The PPV mask can act as a band pass filter, muffling certain frequencies such as the higher order harmonics. Some embodiments to the invention relate to identifying a fundamental frequency of speech and adding back in a missing harmonic using digital signal processor 108. In some embodiments, a missing harmonic can be added to the voice stream at a natural ratio. In this embodiment, the user may train system 150 by pronouncing a sequence of words to system 150. The training is performed with no pressure in the PPV mask. Next the user trains on the same sequence with positive air pressure. The two training sessions are used to identify a natural ratio between the person's fundamental frequency and a harmonic. The ratio can then be used to insert a missing harmonic in the user's voice at the same natural ratio from the fundamental frequency. Optionally the training may be performed at more than one pressure. In one embodiment the training happens within the prescribed therapeutic pressure(s)). The pressure may be an ePAP pressure and greater than 3, 4, 5, 8, or 12 cmH₂O and/or less than 30, 25, 20, or 15 cmH₂O, or within a range of the foregoing endpoints. Alternatively, estimate an average ratio for particular harmonics and add the harmonic based on the estimated ratio.

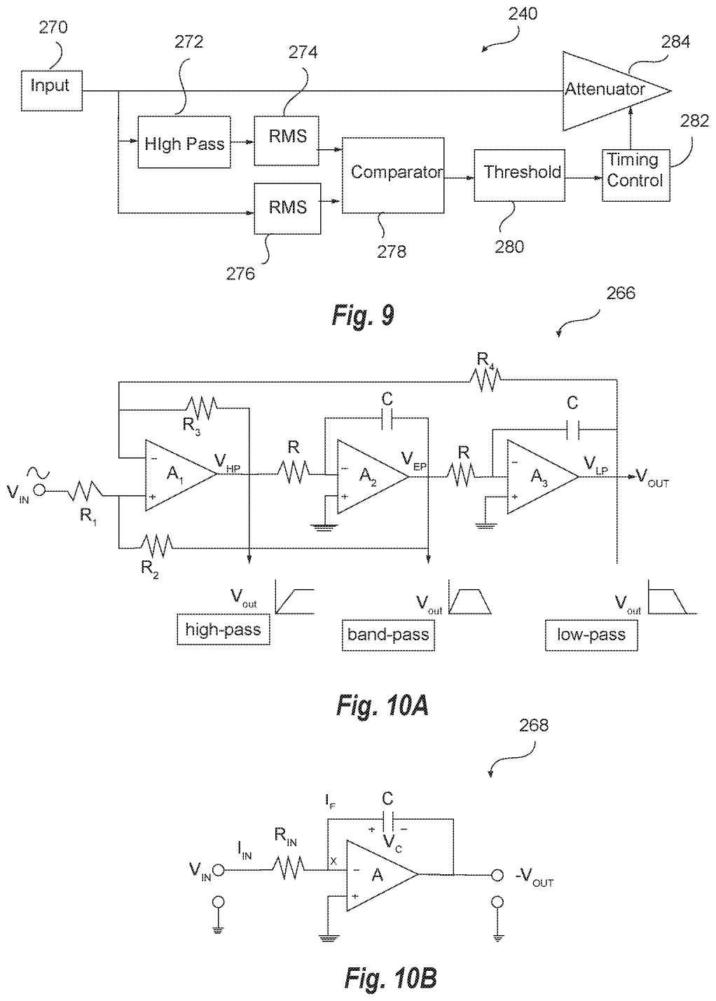

Sibilance Removal. The present invention also relates to performing sibilance removal (block 240). Microphones placed inside the mask using a microphone module have been found to be too sensitive to “s” sounds. FIG. 9 describes circuitry for removing harsh sibilance sounds (sibilance removal 240) while maintaining a natural sounding voice. Voice input from admitter 236 is processed through a high pass filter 272. The filtered signal and the unfiltered signal are then converted to an RMS 274 and RMS 276, respectively and compared in comparator 278. When the filtered RMS 274 is comparable to unfiltered RMS 276 and reaches threshold, sibilance is relatively high and attenuator 284 is activated to attenuate the sibilance. Timing control 282 is used to delay the signal entering the attenuator to allow time for processing to occur.

Sibilance removal module can be used to allow for an improved placement of the mic near the mouth to provide an improved signal to noise ratio. The mic is placed close to the mouth in the mask, which causes disproportionately intense high frequency sounds. The de-esser attenuates the harsh high frequencies. The de-esser is advantageous over EQ because it only removes high signals when they are a problem, which means it can keep other high frequency tones to make the signal sound natural. The de-esser can use RMS (measurement of power average) to compare to power average after a high pass filter. When the two reach a threshold in equivalence, the signal is attenuated (e.g. with a VCA or filter (e.g., mic EQ). The high pass filter frequency can be selected to be between 2 k-10 k frequency. Or alternatively a male voice can be detected and the filter can be set to approximately near 3-6 K or a female detected and set to approximately near 5-8 k.

Some embodiments of the invention include an auto adjust de-esser. The auto de-esser can detect male vs female voice set then set filter accordingly. Alternatively two or more fixed frequencies can be tested and then the system filters above one that is detected to have the most sibilance. Alternatively the system can scan down frequencies until the best filter frequency is found. The auto de-esser can also be carried out in the frequency domain by performing FFT on the amplitude domain and attenuate above the frequency where sibilance is occurring or EQ the signal to remove at the highest sibilance frequency.

Automatic Gain Control. In one embodiment, system 103 includes automatic gain control module 242. A particular level of gain or a range of gain is selected by the user. Module 242 monitors the RMS. When the gain falls outside the particular level or range selected by the user, module 242 adds or subtracts gain to achieve a signal within a desired range of loudness. Thus, if a clinician sets the volume of voice output (e.g., on a speaker box) and the person talks more quietly or louder in a subsequent communication, the automatic gain control module can detect a stronger or weaker signal and automatically adjust the gain up or down to match the user's selected target volume. In an alternative embodiment, automatic gain control can be used to prevent clipping from the pre-amp and maximize the signal to noise ratio. The gain is increased to a level below the max threshold (where clipping occurs). If clipping is occurring the automatic adjustment module adjusts the gain down to below the threshold. The mic preamp may be built into the DSP chip or a stand-alone chip. Automatic gain control is advantageous for NIV microphones because people that are really sick tend to talk quieter and/or have less ability to regulate the loudness of their speech. The automatic gain control allows the clinician to touch the speaker controls less frequently, which reduces potential for contamination and infection of the patient. The process can include: (i) measure short term average power of signal (i.e., window the power) (ii) select target level (i.e., how loud we want it to be around) and a noise level (iii) if signal is below noise level do nothing (you don't want to gain up/amplify noise, (iv) if signal is above noise level and below target level, add gain, and (v) if the signal is above target level gain down.

Power Management. Some embodiments relate to managing power usage of voice amplification system 150. For example, where loudspeaker 107 is battery operated, limiting power usage can increase battery life. Power usage can be minimized in several ways. In one embodiment, system micro uses output from activity detector 230 to power down various components of the system 150. For example, when activity detector 230 detects there is no speech or detects breathing noise, the system micro 160 can turn off amplifier 104. Components of system 150 can also be powered down after a particular amount of time. For example, if no speech activity is detected for a period of time, DSP 108 can be powered down and powering on may require the user to press the “on” button or activity may be detected through an analog band pass filter.

In some embodiments system micro 160 can rapidly turn on and off to perform checks for detecting voice and then power down to save power. When voice is detected, the system microprocessor 160 can turn on the power amplifier. In some embodiments, system micro 160 turns on and off a plurality of times per second.

Some embodiments relate to an analog band pass filter 154 that can be used as a low power monitor for speech detection. In this embodiment, DSP 108 can be powered down when no speech is detected and analog band pass 154 can be used to produce a wake signal that triggers system micro 160 to wake up DSP 108. Analog band pass 154 can be configured to error on the side of producing false positives for speech detection and upon waking, DSP 108 and/or system micro 160 can verify the detected speech. If speech is detected from DSP 108, power amplifier 104 can be powered.

FIGS. 10A and 10B illustrate Analog band pass filters that can be used to perform an analog auto-on feature. Circuit 266 includes an analog band pass filter that isolates frequencies indicative of speech. The analog comparator 268 shown in FIG. 10B can be used to compare frequencies for determining speech vs. noise.

Speaker equalization 244 can be performed on the processed signal to flatten the signal for a particular speaker or enclosure.

In a final step 246, the audio signal is output for amplification and playback on a loudspeaker.

The present invention also relates to digital signal processors that are housed within a microphone module, a speaker housing, a stand-alone housing, or a ventilator. The speaker may be a traditional cone-based speaker, or an exciter.

In some embodiments, the signal processing can be carried out in software on a mobile phone. The microphone may be attached to a mobile phone and processed using the mobile phone processor as described herein with regard to DSP 108. The output may then be transmitted over a mobile phone and/or played on a loudspeaker (e.g., headphones) to a user.

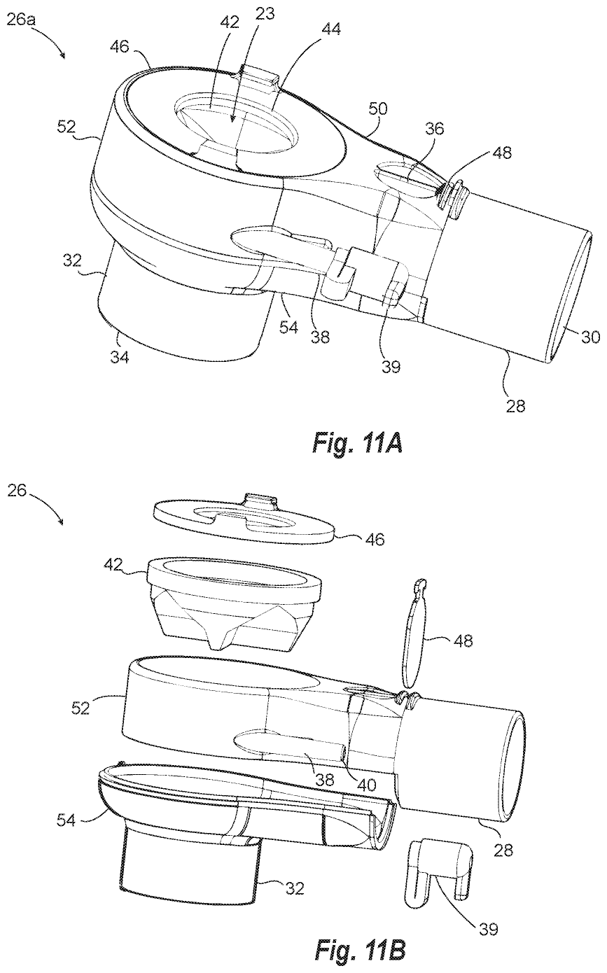

FIGS. 11A-11C illustrate in more detail an elbow suitable for receiving a removable microphone module 100. Elbow 26 includes an elbow body 50 formed from upper housing 52 and lower housing 54. An access valve, such as cross-slit valve 42 (also referred to as “access valve”) is secured to upper housing 52 using a locking ring 46. Elbow 26 also includes an anti-asphyxiation valve that uses a flap 48 to open and close aperture 36. For purposes of this invention, unless otherwise stated or implied, the term “valve” by itself refers to the “access valve” in the access port 23.

Elbow 26 is an air supply connector that includes an air-delivery conduit. The air supply conduit extends between inlet 30 and outlet 34 and includes internal regions 56a, 56b, and 56c (FIG. 11C). Valve 42 is in fluid communication with the air delivery conduit in region 56c. Valve 42 provides access to a wearer's mouth and nose through aperture 44 and region 56c of the conduit.

The air supply conduit provided by valve adapter 26 is configured to deliver pressurized air from a source of positive air pressure (e.g., ventilator 21) to the cavity of the ventilation mask 10. Air pressure in inlet 30 forces flap valve 48 to open to provide fluid communication between regions 56a and 56b. The air flow between region 56a and 56b forces flap 48 upward to close off aperture 36 by seating against seat 58. If air flow stops between regions 56a and 56b, flap 48 drops down to opening to region 56a to prevent air from flowing backwards through inlet 30 (i.e., from region 56a to 56b). Flap 48 prevents asphyxiation by allowing air to be breathed from the ambient (through aperture 36) if the supply of air from the ventilator is interrupted.

Elbow 26 includes a first press-fit connector 28 that serves to fluidly connect a positive pressure air supply hose (not shown) to inlet 30 of elbow 26. A second press-fit connector 32 serves to fluidly connect the outlet 34 of elbow 26 to an inlet in mask body 12. The press-fit connection may be configured to be sufficiently tight that when an appliance is positioned in the adapter (see FIG. 1B) and pulled out of valve 42, the press fit maintains the connection of the air supply connector to the mask. FIG. 1A illustrates a mask with a swivel connector 29 configured on the body of the mask 12. A press fit connector 32 is placed inside of the swivel connector 29 and is configured to be sufficiently tight to deliver air to the mask. The swivel connector has textured finger grips 19 that are used to press on the swivel or rotate the elbow 26.

Elbow 26 preferably swivels relative to mask body 12 such that a hose connected to an elbow 26 can be redirected without torqueing the mask. Any swivel mechanism can be used. The swivel mechanism may be incorporated into a mask body, elbow, or the connection there between.

Connections other than press-fit may be used to connect an elbow 26 to a mask or ventilation system, including non-removable connections, screw fit with screw threads, snap connection, slide in connection with securing ridges, clips, and quick release connections.

FIG. 14 illustrates an embodiment wherein an elbow 526 is configured to form a swivel connection with a mask. A swivel connection portion 300 is shaped to fit in an opening of a PPV mask. The swivel connector 300 is also configured with a sealing rim 304 that will seal with the edges of an opening on the mask. The elbow includes clip connectors 302 that snap into a ridge or mount on the body of the mask to keep the elbow securely fit and sealed on the access port. Release tabs 306 are attached to the clip connector that flex when pressed inward to release the elbow form the mask.

With reference again to FIGS. 11A-11C, elbow 26 may also include a pressure port 40 on stem 38. Pressure port 40 includes a small opening in fluid communication with region 56b that is used to monitor pressure changes in elbow 26. Changes in pressure can be used to detect when the wearer of the mask is inhaling or exhaling. Bi-level pressure ventilators can use the pressure port 40 to provide lower pressure during exhalation and increased pressure during inhalation. Pressure port 40 is not required to be associated with elbow 26, but rather can be placed in mask body 12, tubing between the ventilator and mask, or combinations of these. Pressure port 40 is can be covered with a cap 39 to plug and stop flow when detection is not necessary.

Elbow 26 has an access port 23 with aperture 44 and a valve 42 positioned within the port. Valve 42 may be a seal-sealing valve that uses pressure from the ventilator to close the valve when the access port is clear of an appliance or adapter. The access valve has an open diameter sufficient to perform oral care or insert appliance therethrough with reduced leaking as compared to an access port without the valve and having the same maximum diameter opening. The diameter of the opening in the self-sealing valve (in the fully open position) can be at least 5, 10, 15, or 20 mm (˜0.2, 0.04, 0.06, 0.08 in) and/or less than 50, 40, 30, 25, or 20 mm (˜2, 0.16, 0.12, 0.1, 0.08 in) and/or within a range of the foregoing (in the height and/or width of the opening based on a cross section of the opening). These diameters of opening can be achieved with a valve that will be self-sealing under pressures of at least 4, 5, 8, or 10 cmH₂O and/or less than 30, 25, 20, 15 cmH₂O, or within a range of any of the foregoing endpoints.

In some embodiments, the opening in access valve 42 is provided by one or more slits. The length of the slit may provide the maximum open width. In some embodiments, the valve includes a plurality of slits. In some embodiments, the valve can include 2 slits and the slits may form a cross-slit.

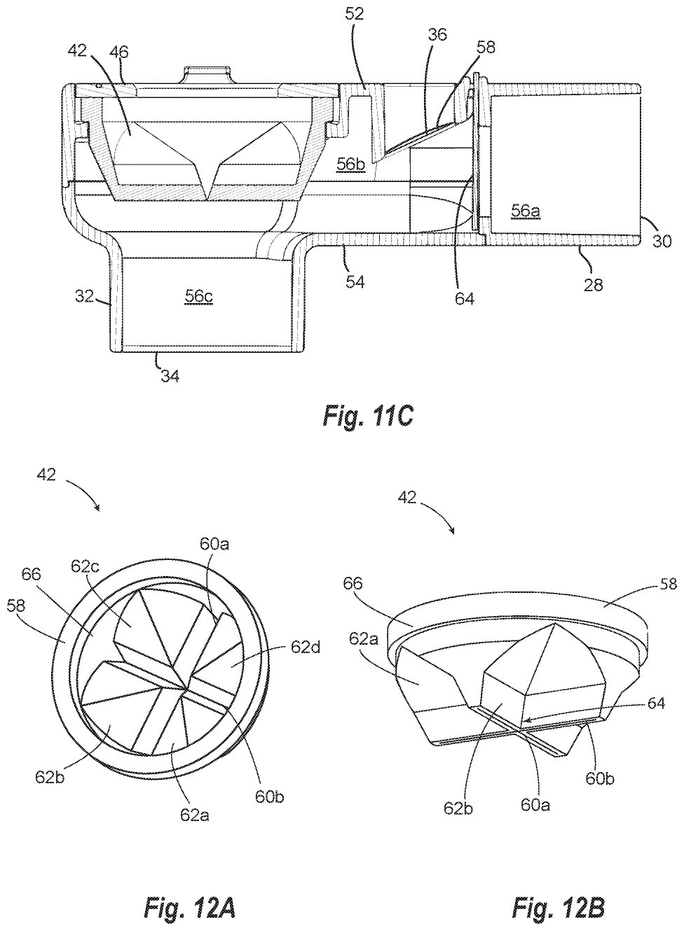

To facilitate self-sealing under pressure, access valve 42 may have inward sloping walls or concavity that the pressure pushes against. Valve 42 may be a duck bill valve or a dome shaped valve. FIGS. 12A and 12B illustrate a duckbill valve with a cross-slit. Valve 42 has a rim 58, support wall 66, and a plurality of leaflets 62a-d. As seen in the top view of FIG. 12A the leaflets are each concave relative to the ventilator pressure side of the valve and form fenestrations at slits 60a and 60b. The leaflets 62 are configured to be pushed open by an appliance or appliance adapter from an outside side of the valve and pushed together by pressure from the inside side of the valve. The duck bill valve is shown with 4 leaflets, but may have a single leaflet (i.e., seals against a rigid wall) but more preferably has at least 2, 3, 4, or more leaflets. As shown in FIG. 12B the concavity of leaflets 62 have a geometry that meets near the center of the cross slit. For example, the concavity of leaflet 62b meets near point 64. When an appliance or adapter is inserted the leaflet 62b is forced out and point 64 moves away from the center cross, thereby opening the valve. Valve 42 can be made from an elastomeric material with shape memory such that upon removing the appliance or adapter, the device recovers at least a portion of its concavity such that the pressure can seal the leaflets.

Where a dome valve is used, the dome may have a tapered thickness that is thin at a center opening and tapers to a greater thickness towards the edges. The taper may include a change of thickness greater than 1.2, 1.5, or 2 times the thickness at a lateral edge of a fenestration/slits as compared to a center edge of a fenestration. The taper may allow the valve to open more easily at the center.

In a preferred embodiment, the valve reverts itself if it becomes inverted (i.e., self-reverting). For purposes of this invention, a self-reverting valve has a material and configuration that causes the valve to return to its self-sealing position when inverted (e.g., an elastomeric material with shape memory). Thus, if an instrument is pulled out of the valve and a leaflet or other component is inverted, the self-reverting valve returns to its self-sealing position once the force is removed. Although not required, the valve may be concave and/or made of a silicone material (or similar polymers, elastomers, isoprene, Nitrile rubber, Butyl rubber, or silicone like material) to facilitate self-reverting. In one embodiment, the valve includes a layer of material at its center that is less than 5, 4, 3, or 2 mm thick. The rim 58 of valve 42 also contributes to the self-reverting feature. The height of the rim above the leaflets and the material extending laterally provide rigidity to the wall buckling when valve 42 is inverted and the material between rim 58 and the leaflets stretching force the leaflets back to their correct position.

The access valve 42 and/or combination of one or more of the access valve 42, anti-asphyxiation valve 40, and mask 12 may be configured to have a leak rate less than 70, 50, 40, 30, or 25 liters per minute (“lpm”) and/or greater than 2, 5, 7, or 10 lpm and/or within a range of the foregoing when the mask is under an air pressure of at least 5, 10, 15, cmH₂O and/or less than 25, 20, or 15 cmH₂O or within a range of the foregoing. For purposes of this invention, the leak rate is measured at a pressure of 5 cmH₂O when measured in accordance with ISO standard 17510 (2015).

In some embodiments, the valve may include a biocompatible lubricant to facilitate insertion of appliances or appliance adapter through the valve. The access valve and/or lubricant may also include an anti-microbial agent (e.g., chlorhexidine). In some embodiments, the valve adapter may have a dust cap that covers the opening to valve 42 the valve is not in use.

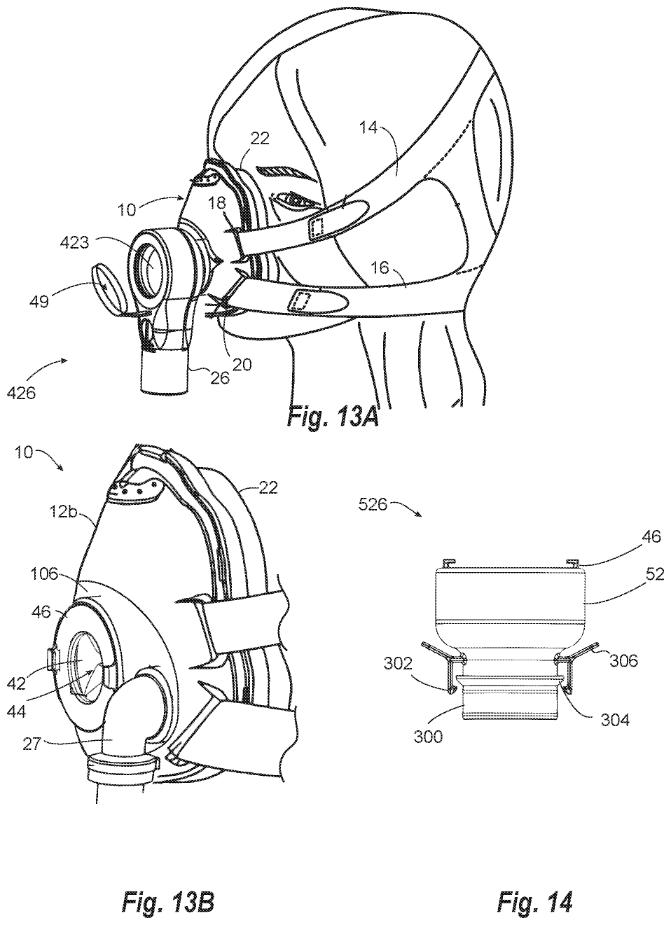

FIG. 13A shows an alternative embodiment with an elbow 426 that does not include the access valve 42 of elbow 26 from FIG. 1A. Elbow 426 includes an aperture 44 configured to receive appliance adapters that will seal the aperture 44 when the adapter is attached and/or placed through the aperture 44 (see FIGS. 7A-C). Because access port 23 of elbow 426 does not have a valve that seals the port when not in use, elbow 426 includes a seal cap 49 that can be placed over or in aperture 44 to prevent air leakage and to maintain air pressure between the mask and the face.

FIG. 13B illustrates an alternative embodiment of a mask 10 having a shell 12b that incorporates the access port 23 into the shell 12b instead of the elbow connector. Shell 12b has an elbow connector 27 separate from access port 23. Elbow connector 27 supplies pressurized air to the mask and may have any features known in the art for elbow connectors used on PPV masks. Similar to mask 10 of FIG. 1A, valve 42 seals access port 23 using positive pressure in mask 10. Placing access port 23 in the mask separate from the elbow connector allows elbow 27 to be smaller than elbow 26 of FIG. 1A.

The access port in shell 12b may also be configured without a valve as shown in access port 423 of FIG. 13A. In addition, access ports 23 (with or without a valve) can be placed anywhere on shell 12b that allows direct external access to the mouth or nose of the patient (i.e., access to the mouth or nose through the mask). In some embodiments, a microphone module and/or microphone can be incorporated into a shell of the mask with the microphone elements positioned inside the mask. The microphone elements may be permanently positioned in the shell of the mask.

FIG. 13B also illustrates one embodiment showing a mask body 12b with a flexible portion 106 that is more flexible than the material of the adjacent portion of mask body 12b. The flexible portion 106 provides greater articulation and movement for appliance adapters placed through the access valve 42 as well as support and flexibility in maintaining a seal around the face created by the cushion 22. The flexible portion 106 of shell 12b can also be incorporated into mask body of the embodiments shown in FIGS. 1A and 13A. In an alternative embodiment, the swivel connector 29 of FIG. 1A or 13A can be configured to be more flexible than the body of the mask 12. Flexible elbow connectors are further described in U.S. Pat. No. 8,302,605 which is hereby incorporated herein by reference. In yet another embodiment, the access port may be an iris valve such as the valve described in US2003/047189 to Kumar, which is hereby incorporated by reference. The present invention also includes methods for using any of the other appliances described herein.

Applicants co-pending US Provisional Patent Application Nos. 62/568,314, filed Oct. 4, 2017 and 62/612,303, filed Dec. 29, 2017, and Applicant's PCT application No. PCT/US2016/039117, filed Jun. 23, 2016, and PCT/US2017/060480, Filed Nov. 7, 2017 are each hereby incorporated herein by reference in their entirety.

The present invention can be incorporated into various masks and/or adapters using a variety of materials. Examples of positive air pressure masks that can be adapted to include a valve according to one embodiment of the invention are illustrated in U.S. Patent Application publications US2009/0194111 to Fu et al and US2010/0116276 to Bayasi. The method of the present invention are not limited to the novel masks and mask systems described herein. For example, the methods can be carried out using the mask of U.S. Pat. No. 6,792,943 or 8,365,734 to Lehman. The foregoing patents and applications are incorporated herein by reference for their teachings of masks and components that can be used in combination with the features of the present invention and their use for carrying out the methods described herein.

We claim:

1. A positive pressure ventilation (PPV) microphone module, comprising:
a microphone housing defining an adapter configured to be removably inserted into a port of a PPV mask and having a surface that forms a seal with the port when inserted therein, the microphone housing having a distal portion and a proximal portion, wherein the distal portion of the microphone housing is distal to the seal and configured to be inserted through the port and position an oral end thereof within the PPV mask, and wherein the proximal portion is proximal to the seal and configured to be exposed to ambient pressure;
one or more mic elements positioned on the oral end of the microphone housing and configured to receive speech from a person wearing the PPV mask and generate a mic signal;
electrical circuitry within the microphone housing configured to receive the mic signal from the one or more mic elements, the electrical circuitry exiting the microphone housing through a wall of the proximal portion of the microphone housing.

2. The PPV microphone module as in claim 1, wherein the electrical circuitry includes a connector positioned in the wall and provides the exit from the microphone housing, the connector configured to removably connect to a data cable for transmitting the mic signal to an audio processing system.

3. The PPV microphone module as in claim 1, further comprising a loudspeaker disposed in the microphone housing.

4. The PPV microphone module as in claim 1, wherein the distal portion forms a tubular structure configured to be slidably received in the port and wherein the oral end of the microphone housing defines a cavity with a distally facing opening and wherein the one or more microphone elements are disposed in the cavity.

5. The PPV microphone module as in claim 4 wherein the one or more mic elements have a max sound pressure level of at least 105 dB and a dynamic range of at least 85 dB.

6. The PPV microphone module of claim 4 wherein the one or more microphone elements are disposed in the cavity and an attenuator is positioned between the one or more microphone elements and the opening, the attenuator configured to reduce sound pressure levels by at least 3 dB across the attenuator.

7. The PPV microphone module as in claim 6, wherein the attenuator comprises foam with a density of at least 2 lb-ft³.

8. A PPV microphone system, comprising:
the PPV microphone module of claim 1;
an audio processing system including (i) a system microcontroller, (ii) an input configured to receive the mic signal from the PPV microphone module (iii) a power amplifier configured to use the mic signal to produce an amplified signal suitable for powering a loudspeaker and (iv) an output for providing the amplified signal to a loudspeaker.

9. The PPV microphone system as in claim 8, wherein the audio processing system is disposed within a second housing and the microphone module is connected to the audio processing system through a cable.

10. The PPV microphone system as in claim 9, further comprising a loudspeaker positioned within the second housing.

11. The PPV microphone system as in claim 9, further comprising a loudspeaker positioned within the microphone housing.

12. The PPV microphone system as in claim 8 wherein the cable removably connects to first and second connectors in the microphone housing and the second housing, respectively.

13. A method for using the microphone system of claim 8, comprising:
providing a PPV mask connected to a ventilation circuit supplying at least 3 cmH₂O of positive pressure, the PPV mask having an access port configured to receive the microphone module and form a PPV seal therewith;
inserting the microphone module into the port and forming a PPV seal therewith;
with the PPV mask under a pressure of at least 3 cmH₂O, generating a speech signal using the microphone module, amplifying the speech signal using the audio processing system, and providing the amplified speech signal to a loudspeaker.

14. The method of claim 13, wherein the port includes a valve that seals under pressure from the ventilator and has a diameter in a range from 10 mm to 50 mm.

15. The method of claim 13, wherein the port includes a valve that self-seals under pressure from the ventilator.

16. The method of claim 14, wherein the port is positioned within an elbow connector.

17. The method of claim 13, wherein generating the speech signal is carried out with the one or more mic elements positioned in front of the patient's mouth at a distance less than 50.8 mm (2 inches).

18. A positive pressure ventilation (PPV) microphone system, comprising:
(i) a PPV microphone module including:
a microphone housing defining an adapter configured to be slidably and removably inserted into a port of a PPV mask and having a surface that forms a seal with the port when inserted therein, the microphone housing having a distal portion and a proximal portion, wherein the distal portion of the microphone housing is distal to the seal and configured to be inserted through the port and position an oral end thereof within the PPV mask, and wherein the proximal portion is proximal to the seal and configured to be exposed to ambient pressure;
one or more mic elements positioned on the oral end of the microphone housing and configured to receive speech from a person wearing the PPV mask and generate a mic signal; and
electrical circuitry within the microphone housing configured to receive the mic signal from the one or more mic elements, the electrical circuitry including a connector that exits the microphone housing through a wall of the proximal portion of the microphone housing;
(ii) an audio processing system disposed in a second housing and including:
a system microcontroller;
an input configured to receive the mic signal from the PPV microphone module;
a power amplifier configured to use the mic signal to produce an amplified signal suitable for powering a loudspeaker; and
an output for providing the amplified signal to a loudspeaker; and
(iii) a cable that removably connects to the connector of the microphone module and is configured to provide the mic signal to the audio processing system through the input.

19. The PPV microphone module as in claim 18, further comprising a loudspeaker disposed in the microphone housing.

20. A method for using the microphone system of claim 8, comprising:
providing a PPV mask connected to a ventilation circuit supplying at least 3 cmH₂O of positive pressure, the PPV mask having an access port configured to receive the microphone module and form a PPV seal therewith, wherein the access port is positioned in an elbow connector of the mask and includes a cross slit valve that self-seals under ventilator pressure;
inserting the microphone module into the port and forming a PPV seal therewith;
with at least 3 cmH₂O positive pressure within the PPV mask, generating a speech signal using the microphone module, amplifying the speech signal using the audio processing system, and providing the amplified speech signal to a loudspeaker.
